(19) United States
(12) Patent Application Publication
(10) Pub. No.: US 2004/0072360 A1
(43) Pub. Date: Apr. 15, 2004

(54) Nitric oxide (no) detector

(76) Inventors: Ron Naaman, Nes Ziona (IL); Dmitry Shvarts, Kiryat-Ono (IL); Dengguo Wu, Huhnot (CN); David Cahen, Rehovot (IL); Avner Haran, Nes Ziona (IL); Aharon Benshafrut, Ramat Gan (IL)

Correspondence Address: BROWDY AND NEIMARK, P.L.L.C., 624 NINTH STREET, NW, SUITE 300, WASHINGTON, DC, 20001-5303, US

(21) Appl. No.: 10/250,992

(22) PCT Filed: Jan. 17, 2002
(86) PCT No.: PCT/IL02/00045

(30) Foreign Application Priority Data
Jan. 17, 2001 (IL) 140949

(51) Int. Cl. G01N 27/02

(52) U.S. Cl. 436/116; 422/88; 422/98

(57) ABSTRACT

A semiconductor device (FIG. 1) is provided for the detection of nitric oxide (NO) molecules in gaseous mixtures, in biological fluids and in aqueous solutions. The device is a molecular controlled semiconductor resistor (MOCSER) of a multilayered GaAs structure to which top layer a layer of multifunctional NO-binding molecules are adsorbed. The sensitivity of the semiconductor device towards NO is independent of mixture composition. Nitric oxide concentrations of as low as 10 ppb NO were detected in mixtures containing various contaminants.

### FIELD OF THE INVENTION

The present invention relates to nitric oxide (NO) detectors and more specifically to an NO detector based on molecular controlled semiconductor resistors.

### BACKGROUND OF THE INVENTION

Nitric oxide is one of the most extensively investigated molecules in the fields of inorganic and bioinorganic chemistry. The study of the molecule in biological systems received a renewed interest because of its role in a myriad of biological events. It is probably correct to state that nitric oxide is involved in practically every common pathophysiological event by virtue of its importance in the normal maintenance of many important physiological phenomena ranging from the protection of the heart, stimulation and regulation of brain functions and vascular tone, to responding to vascular injuries and pulmonary diseases. The 1998 Nobel Prize in Medicine was awarded jointly to Robert F. Fuchogott, Louise J. Ignarro and Ferid Murad for their discoveries concerning“Nitric Oxide as a Signaling Molecule in the Cardiovascular System”.

The production of NO in the human body proceeds via one of two pathways: an enzymatic and a nonenzymatic pathway. The enzymatic pathway involves the action of the nitric oxide synthases (NOS) on the amino acid arginine with the production of the metabolites citrulline and NO. This five-electron oxidation reaction requires reduced pyridine nucleotides, reduced biopteridines and calmodulin. In the bloodstream, NO binds primarily to hemoglobin, being then converted to N₀₃⁻ and eliminated in the urine with a half-life of 5 to 8 hours.

N₀₃⁻ from food and inhaled NO is concentrated in the saliva and converted to nitrite by bacteria on the surface of the tongue. When saliva is swallowed, the nitrite is converted to NO in the stomach, providing defense against swallowed microorganisms. This NO production was demonstrated in the stomach, on the surface of the skin, in infected nitrite-containing urine and in the ischemic heart (Weitzbarg et al., 1998).

Since the formation of NO is connected with several pathophysiological events, the measurement of NO is important for the characterization of important biological functions during which a change in the measured levels of NO produced may indicate the existence of a disease or pathogenesis event. One example for such a phenomena is the measurable change in NO production in exhaled air during airway inflammation in asthma and other diseases. Measurements of exhaled nitric oxide (ENO) are regarded as a marker for the airway inflammations as the concentration of ENO is nearly tripled in the pathogenesis of asthma. As exhaled NO is not increased during bronchospasm in the absence of coexisting inflammation, it serves to differentiate between the components of asthma and thereby helps to direct to the appropriate medication (Hunt et al., 2000; Kissoon et al., 1999).

In addition to biological events, it is known that oxides of nitrogen (NOₓ) originating from motor vehicles, fossil fuel and power plants are major pollutants that affect human health and the ecology. Primary emissions are CO, NO and unburnt hydrocarbons. It wasn't until the 1990s that NO emissions from cars were recognized as the major cause of environmental pollution (Menil et al., 2000). Furthermore, the nitrogen oxides (NO₂ or NO) are a source of ozone, which causes an increase of smog in large cities. This process, which occurs via solar irradiation and photolytic decomposition of NO₂, is a source of acid rain. At the same time, NO in the atmosphere reacts with ozone to replenish the reacting NO₂, and the cycle continues.

Monitoring the emission of these pollutants, their transport in the atmosphere, and their degradation to second-generation pollutants is crucial. Direct monitoring of NO in the emissions of combustion engines requires a sensor capable of sustaining high temperatures, low concentrations of NO (100-1000 ppm) and corrosive medium containing oxygen and water vapor. Under these conditions, the nitrogen oxide (NOₓ) mixtures contain mainly NO.

The present monitoring techniques of nitrogen oxide mixtures are expensive, the measuring devices are bulky and their use is therefore unpractical and problematic. Efforts have been concentrated on developing many kinds of NOₓ sensors such as electrochemical sensors which utilize solid electrolytes, thin film superconductor type sensors, semiconductor oxide type sensors using SnO₂, ZnO, WO₃, and TiO₂ oxide ceramics or thin films, etc. Using SnO₂ as sensing material, the concentration of gaseous NO was determined to levels as low as 10 ppm whereas with solid electrolytes only concentrations in the order of 10³ ppm NO were detectable (Kudo et al., 2000; Becker et al., 2000; Wang et al., 2000).

Nitric oxide (NO) is a small, uncharged, paramagnetic molecule, existing in gas or liquid phases. In the gas phase the molecule is stable, compared with a short half-life of between 5 and 15 seconds measured in biological media. Its diffusion constant in physiological medium measured at 3300 μm²/s is very similar to that in water. The solubility of NO in hydrophobic solvents is nine times greater than in aqueous solutions, which makes NO an excellent transmitter agent and inflictor of cellular damage, acting without the necessity of specific export mechanism such as vascular secretion. NO reacts with oxygen species and metals to yield oxidized products such as nitrites and nitrates, NO₂⁻ and NO₃⁻, respectively.

Several methods for detection of NO in solution and in the gas phase have been developed in recent years for diagnostic or environmental purposes. The fact that NO is very reactive in biological tissues makes its direct quantification very complex and many measurements, therefore, relied on indirect methods, determining levels of NO metabolites such as nitrite and nitrate anions or NO precursors such as citrulline instead of NO itself.

The most frequently used method to measure the stable nitrite end product is based on purple azo dye that was found by Griess more than 100 years ago to recognize nitrite. In this method, the nitrite anion binds to N-(1-naphthyl)-ethylenediamine (NED) to produce a purple dye. Screening the dye-containing solutions by light absorption at 550 nm produces the appropriate emission (Schulz et al., 1999). This method does not detect the second metabolite of nitric oxide, the nitrate anion NO₃⁻, thus limiting the detection to only a fraction of the volume of NO produced. However, the reduction of the nitrate anion to the nitrite is usually achieved using bacterial nitrate reductase or reducing metals such as cadmium. The detection limit for the nitrite anion in biological fluids, under the Griess method, is 1.0-1.5 μM (30-45 ppb), with a reaction time of about 20 minutes. A similar method utilizing 2,3-diaminonaphthalene (DAN) as the nitrite-binding substrate was determined to be 10 times more sensitive than the conventional technique and at least 50 times more sensitive for determining nitrite concentrations in sera or aqueous solutions (Kojima et al., 2000; Casey et al 2000).

For directly measuring NO levels in vivo, 1,2-diaminoanthraquinone (DAQ) was found suitable. It produces a red-fluorescent precipitate when in contact with NO. This compound was used to detect changes in NO levels in rat retinas after injury to the optic nerve.

In another indirect method, quantification of citrulline instead of NO was pursued. However, levels of the amino acid in sera and urine are not good indicators of NO production. In cultured cells, the presence of citrulline is primarily due to NO synthase enzyme (NOS) activity. Measurements indicated that the citrulline levels were not stoichiometrically equivalent to total NO levels as measured by a series of different methods (Marzinzig et al., 1997).

Other methods for NO identification and quantification include electrochemical, fluorescent and transistor-based methods. In one of these methods, the NO is trapped by nitroso compounds or reduced hemoglobin forming stable species that can be quantified by EPR (electron paramagnetic resonance) with a detection limit of 1 μM (30 ppb). In another method NO levels in the gas phase are detected by reaction with ozone, producing chemilumiescence, with a detection limit of 20 nM (ppt concentration). Recent electrochemical methods offer the possibility to measure even lower concentrations of NO (at the pM limit) in intact tissues and single cells (Hunt et al., 2000; Kotake et al., 1999).

Presently existing NO sensors have been manufactured for bedside treatments in hospitals and medical laboratories for the purposes of treatment and/or diagnostics. These sensors are based on the above-mentioned methods of analysis and thus suffer from several basic disadvantages such as low S/N ratios, cross sensitivity to other components in the test medium, expensive and time-consuming operational steps and inaccurate quantification of NO or its metabolites due to NO's short half-life.

Several methods and devices for measurement of NO in lung conditions, in the oral cavity, in the urogenital tract and in the intestines were described in the U.S. Pat. Nos. 5,447,165, 5,922,610, 6,038,913, 6,063,027 and 6,099,480, and in the PCT Publications WO 09843539 and WO 09939100.

PTC Publication No. WO 98/19151 (Cahen et al., 1998), of the same applicants of the present application, herein incorporated by reference as if herein described in its entirety, describes a hybrid organic-inorganic semiconductor device and sensors based thereon, said device characterized by being composed of:

(1) at least one layer of a conducting semiconductor;

(2) at least one insulating layer;

(3) a multifunctional organic sensing molecule directly chemisorbed on one of its surfaces, said multifunctional organic sensing molecule having at least one functional group that binds to the said surface of the electronic device, and at least one other functional group that serves as a sensor; and

(4) two conducting pads on the top layer making electrical contact with the electrically conducting layer (1), such that electrical current can flow between them at a finite distance from the surface of the device.

These Molecular Controlled Semiconductor Resistors, also designated MOCSER, are described in said WO 98/19151 as light or chemical sensors.

### SUMMARY OF THE INVENTION

It has now been found, according to the present invention, that a device such as that described in WO 98/19151 can serve as a sensor for nitric oxide gaseous as well as dissolved in biological fluids and in solution, and can specifically detect NO concentrations in gaseous, biological, and aqueous media.

The present invention thus relates to a semiconductor device (MOCSER) for the detection of nitric oxide (NO), said device being composed of:

(i) at least one layer of a conducting semiconductor;

(ii) at least one insulating or semi-insulating layer;

(iii) a layer of multifunctional organic molecules capable of binding nitric oxide, said molecules being directly bound to the surface of an upper layer which is either a conducting semiconductor layer (i) or an insulating or semi-insulating layer (ii); and

(iv) two conducting pads on the upper layer making electrical contact with the conducting semiconductor layer (i), such that electrical current can flow between them at a finite distance from the surface of the device.

The multifunctional organic layer (iii) is composed of molecules that can bind NO such as, but not being limited to vicinal diamines, metalloporphyrins, metallophthalocyanines, and iron-dithiocarbamate complexes. In order to bind directly to the surface of the upper layer these molecules should contain at least one functional group as the surface binding group (SG) such as, but not being limited to, carboxyl, thiol, acyclic sulfide, cyclic disulfide, hydroxamic acid, trichlorosilane or phosphate groups. When the original molecule that binds NO does not contain a functional group that binds to the surface, one or more desired functional groups can be added to said organic molecules by methods well known in the art of chemical synthesis.

Examples of vicinal diamines that bind NO and can be used according to the invention are, without being limited to, 2,3-diaminonaphthalene, 1,2-diaminobenzene, 1,2-diaminoanthraquinone or aminotroponiminate (see Appendix) that are substituted at the ring or at one of the amino groups with at least one suitable surface binding group as defined above, or the amino group is linked through an aliphatic, aromatic or araliphatic spacer to such a surface binding group. Examples of such spacers with their length and composition are shown in the Appendix herein, but it is evident to any one skilled in the art that spacers of different length and composition can be used according to the invention.

Examples of metalloporphyrins and metallophthalocyanines that bind NO and can be used according to the invention are, without being limited to, those containing as central metal atoms Fe, Co, Ni, Zn, Mk, Cu, Ru, V, Pb or Cr. Many of the natural porphyrins contain functional groups such as carboxyl groups on the side chains. For example the metalloporphyrins derived from hematoporphyrin or protoporphyrin IX (see Appendix) such as hematin (ferriprotoporphyrin basic), heme (ferroprotoporphyrin), hemin (ferriprotoporphyrin chloride) and cobaltic protoporphyrin IX chloride contain at positions 2 and 18 two propionic acid side chains, namely a carboxyl group linked through a spacer—(CH₂)₂— in each position. When such functional groups do not exist in the natural molecule, desired groups consisting of a spacer terminated with one of the surface-binding groups can be inserted at one of the peripheral carbon atoms by methods well known in the art of chemical synthesis. The same procedures can be used to prepare suitable metallophthalocyanines.

The iron-dithiocarbamate complexes that can be used according to the invention bind NO through the iron center and to the surface of the device through a surface-binding group as mentioned above having a spacer ejected from the nitrogen center. The spacer may be aliphatic, aromatic, or a combination thereof, and of varying lengths. The dithiocarbamate complex may be symmetric or unsymmetric.

The invention further relates to an array of semiconductor devices, wherein each device in the array is covered with a monolayer consisting of a different NO-binding molecule. Said array may optionally further contain other devices carrying monolayers of compounds capable to bind to contaminants of NO mixtures such as CO, oxygen, etc.

In another aspect, the present invention relates to a method for the detection and measurement of nitric oxide, which comprises:

(i) exposing a semiconductor device or an array of devices according to the invention to a sample containing NO; and

(ii) monitoring the presence of NO in the sample and determining its concentration according to the change in the current measured at a constant electric potential applied between the two conducting pads.

The sample containing NO may be gaseous, aqueous or mixtures thereof. In one embodiment, the sample is a biological fluid such as exhaled air, endogenous gaseous NO of the urogenital tract or from the lumen of the intestines. When the sample is exhaled air, the method is suitable for evaluating lung conditions for example in asthma patients. Measurement of NO from the urogenital tract e.g. from the bladder, urethra, uterus and oviducts, or from lumen of the intestines, permits to evaluate inflammatory conditions in these organs.

### BRIEF DESCRIPTION OF THE DRAWINGS

The present invention will be understood and appreciated more fully from the following detailed description, taken in conjunction with the examples and drawings, in which:

Figs. 1a-b depict schemes of the MOCSER device of the present invention: 1a depicts the layered structure and 1b the layout.

FIG. 2 represents the response of the MOCSER device, covered with a mixed monolayer of hemin and benzoic acid molecules to various concentrations of NO dissolved in physiological media. The insert presents the calibration curve for the device where the NO concentration in the media is correlated with the time constant measured.

FIGS. 3a-b show measurement of NO produced from brain tissues as measured by a MOCSER immersed in the artificial cerebrospinal fluid (ACSF) at a distance of less than 1 mm from the brain slice, in the presence (FIG. 3a) and absence (FIG. 3b) of H₂O₂.

FIGS. 4a-b demonstrate the sensitivity of the sensor to NO. FIG. 4a depicts the response of the device to different concentrations of NO gas in dry air. FIG. 4b presents the calibration graph obtained both in nitrogen (open circles) and dry air (filled stars) as a diluting gas. Insert to FIG. 4b shows the low-concentration range of the calibration graph more clearly.

FIGS. 5a-b show the reversibility of the device: a) NO dissolved in aqueous media, b) NO gas in air.

FIG. 6 shows the sensitivity towards NO as calculated from results.

FIGS. 7a-c demonstrate that the effect of exposure of the sensor to gases other than NO is minimal. FIG. 7a shows exposures to CO and O₂. FIG. 7b and FIG. 7c show the response of the sensor to NO after pre-exposure to carbon monoxide or oxygen followed by purging.

### DETAILED DESCRIPTION OF THE INVENTION

According to the present invention, there is provided a device for the detection of nitric oxide being a molecular controlled semiconductor resistor, herein designated MOCSER, said device being composed of one or more semi-insulating layers, one conducting semiconductor layer, two conducting pads, and a layer of multifunctional organic molecules, characterized by:

(i) said conducting semiconductor layer being on top of one of said insulating or semi-insulating layers;

(ii) said two conducting pads being on both sides on top of an upper layer which is either said conducting semiconductor layer or another of said insulating or semi-insulating layers, making electrical contact with said conducting semiconductor layer; and

(iii) said layer of multifunctional organic molecules consists of molecules capable of binding nitric oxide, said molecules being directly bound to the surface of said upper layer, between the two conducting pads.

The multifunctional organic molecules that bind NO are molecules such as vicinal diamines, metalloporphyrins, metallophthalocyanines, and iron-dithiocarbamate complexes that have one or more aliphatic, aromatic or araliphatic side chains terminated by a functional group such as carboxyl, thiol, acyclic sulfide, cyclic disulfide, hydroxamic acid and trichlorosilane, said functional groups being directly bound to the surface of said upper conducting semiconductor layer or insulating or semi-insulating layer.

The device according to the invention serves as an amplifier, which translates the NO concentration on its surface into change in the electrical current. Binding of NO to the sensing multifunctional molecules results in a change of the charge distribution, followed by change in the electrical current, as described previously for different molecules (Gartsman et al., 1998; Vilan et al., 1998).

In one embodiment, the semiconductor device of this invention is composed of one or more insulating or semi-insulating layers (1), one conducting semiconductor layer (2), two conducting pads (3), and a layer of at least one capable of binding NO (4), characterized in that: said conducting semiconductor layer (2) is on top of one of said insulating or semi-insulating layers (1), said two conducting pads (3) are on both sides on top of an upper layer which is either said conducting semiconductor layer (2) or another of said insulating or semi-insulating layers (1), making electrical contact with said conducting semiconductor layer (2), and said layer made of at least one compound capable of binding NO is adsorbed on the surface of said upper layer, between the two conducting pads (3).

The semiconductor of layer (2) of a MOCSER of the invention may be a semiconductor selected from a III-V and II-VI material, or mixtures thereof, wherein III, V, II and VI denote the Periodic Table elements III=Ga, In; V=As, P; II=Cd, Zn; VI=S, Se, Te. In preferred embodiments, the conducting semiconductor layer (2) is a doped n-GaAs or doped n-(Al,Ga)As, doped preferably with Si.

In another embodiment, the one or more insulating or semi-insulating layers (1) of a device of the invention, that may serve as the base for the device, is a dielectric material selected from silicon oxide, silicon nitride or from an undoped semiconductor selected from a III-V and a II-VI material, or mixtures thereof, wherein III, V, II and VI denote the Periodic Table elements III=Ga, In; V=As, P; II=Cd, Zn; VI=S, Se, Te and is preferably undoped GaAs or (Al,Ga)As substrate.

In one preferred embodiment, the MOCSER of the invention is based on a GaAs/(Al,Ga)As structure. According to this preferred embodiment, there is provided a MOCSER wherein said conducting semiconductor layer (2) of doped n-GaAs is on top of a semi-insulating layer (1) of (Al,Ga)As which is on top of another semi-insulating layer (1) of GaAs, and on top of said conducting semiconductor doped n-GaAs layer (2) there is a semi-insulating undoped GaAs layer (1) to which is attached said layer of at least one compound capable of binding NO (4).

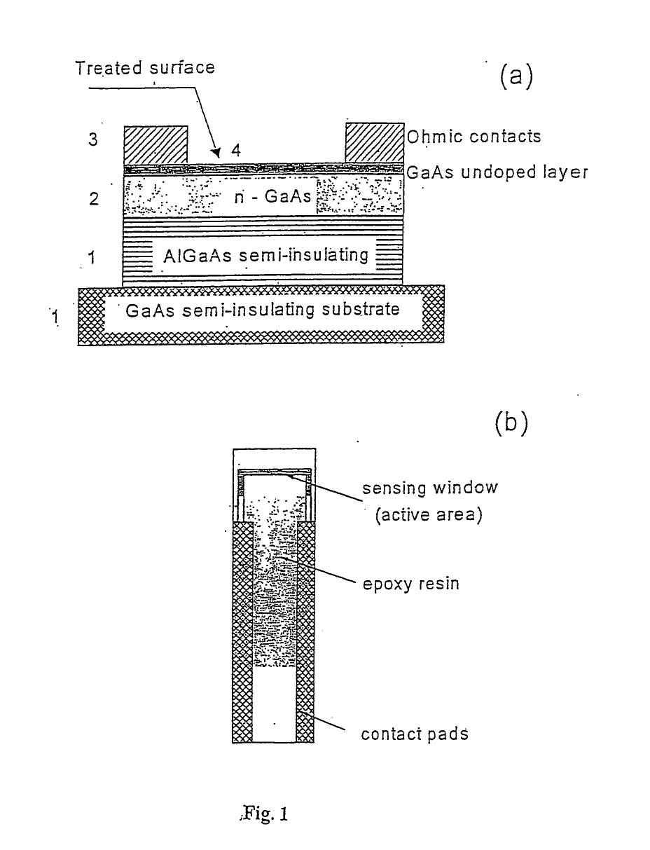

A MOCSER according to the invention was developed as disclosed in WO 98/19151 as a multilayered GaAs based device as depicted in FIG. 1 which contains a conducting n-doped GaAs upper layer (active layer of 450-500 Å, doped to concentration of 4-7E17 cm⁻³) that is close to the surface. This active layer lies between semi-insulating layers, e.g. an undoped semi-insulating uppermost GaAs layer (50-100 Å) and a semi-insulating AlGaAs layer (of 1500-4000 Å) above a GaAs semi-insulating substrate, connected to two ohmic contacts, e.g. AuGeNi. The MOCSER will preferably be rinsed in organic solvents and treated in ozone cleaning system prior to use.

According to this same preferred embodiment, there is further provided a MOCSER wherein said conducting semiconductor layer (2) of doped n-(Al,Ga)As is on top of an insulating layer (1) of undoped GaAs which is on top of a semi-insulating layer (1) of GaAs, on top of said conducting semiconductor doped n-(Al,Ga)As layer (2) there is a semi-insulating undoped (Al,Ga)As layer (1) on top of which there is an upper undoped GaAs semi-insulating layer (1), and said monolayer of at least one compound capable of binding nitric oxide (4) is attached to the upper undoped GaAs semi-insulating layer (1).

The sensing metalloporphyrin or other similar organic compound capable of binding NO making-up the monolayer will vary according to the purpose of the detection and the medium or environment in which the nitric oxide is to be tested.

Examples of the various applications of the MOCSER as a sensor for nitric oxide, without being limited to: (1) detection of NO in exhaled air for monitoring asthma and/or other airway inflammation and/or gastric activity; (2) detection of NO in polluted air; (3) in-vitro detection of NO in various physiological media, resulting from NO-producing living cells; (4) in-vivo detection of NO in physiological medium and in living cells, for the purpose of measuring metabolic activity, and/or toxicity, and for the diagnosis of heart diseases, circulatory shock and cancer.

The invention also relates to an array of semiconductor devices (MOCSERs) as described above, wherein at least one device contains the NO-binding compound and at least one of the remaining devices in the array is adsorbed with a different selective organic molecule which selectively binds contaminants present along with the nitric oxide in the tested medium. Examples of such contaminants are carbon monoxide, oxygen, inorganic salts and other organic and inorganic molecules present in exhaled air, bodily fluids, biological solutions and other media. These molecules are well known in the art.

In one preferred embodiment, at least one of said MOCSERs in the array is covered with a monolayer of molecules that bind NO and at least one of the other devices contains a molecule that binds selectively the contaminating species, e.g. CO and/or O₂. The response of each individual MOCSER is measured, recorded and then processed to extract the signal produced by the NO-binding molecules.

According to the present invention, a device for detection of Nitric Oxide (NO) is provided that is based on a MOCSER structure, preferably of a GaAs/(AlGa)As device, where on top of one of its surfaces a monolayer of NO-binding organic molecules is adsorbed. A current flows through the device when voltage is applied between its two electrodes. When the adsorbed monolayer of NO-binding molecules interacts with NO molecules, present in the tested medium, the charge distribution in the binding molecules changes. The change in the charge distribution affects the current flowing through the device.

The concentration of the NO in the medium can be monitored as correlated from the electronic response of the device: the higher the NO concentration, the faster/higher is the observed change in the MOCSER's current.

This invention will be fully appreciated from the following detailed description and examples taken in conjunction with the drawings.

FIG. 1 depicts schematically an NO detector according to this invention based on a field effect transistor (FET) in which two electrodes are used. This FET-like device structure has a semi-insulating, undoped buffer (Al,Ga)As layer (1) on top of a semi-insulating GaAs substrate (1), a thin layer of conducting semiconductor n-GaAs (2) (the active layer) on top of the semi-insulating (Al,Ga)As layer (1), a protective upper thin layer of undoped semi-insulating GaAs layer (1) covering the conducting semiconductor n-GaAs layer (2), and a monolayer (4) of a NO-binding compound such as a metalloporphyrin adsorbed on the undoped GaAs surface (1). Two conducting AuGeNi electrodes (3) serve as electric contacts. These are the two ohmic contacts—source and drain, connected to the n-doped GaAs active layer that lies between the semi-insulating layers.

This molecular controlled semiconductor resistor (MOCSER) is highly sensitive to chemical changes on its surface. The molecules that are adsorbed on the GaAs surface change the surface potential, which affects the resistance of the MOCSER. The MOCSER also has a short response-time (Vilan et al., 1998) and its operation is very simple.

The detection of nitric oxide (NO) and its quantification is a very important tool in the diagnosis of diseases and environmental pollution.

The measured binding (affinity) constants of NO to the metallic heme centers reflects the stronger interactions of the NO group as compared with that of CO. Direct addition of NO gas or of an aqueous solution of NO to metalloporphyrins or heme appears to be the most widely used method for the preparation of nitrosyl metalloporphyrins or nitrosyl-hemes. These have been studied extensively in past years as better understanding of the vital role of NO in mammalian life was realized.

In one preferred embodiment of the invention, the NO-binding compound is a metalloporphyrin. The combination of the sensitivity of the MOCSER and the affinity of the organic metalloporphyrins layer towards the NO molecule, with high selectivity as compared with carbon monoxide, carbon dioxide, nitrogen dioxide, oxygen, nitrogen and water are the basic principles behind the present invention. The greater affinity of the metalloporphyrins-covered MOCSER to NO as compared to the contaminating species such as CO, allows the detection of NO in complex mixtures such as exhaled air. As a result of the reaction between the monolayer of metalloporphyrins and the NO molecules, producing a monolayer of nitrosyl porphyrins, a small change in the conductivity of the MOCSER will be induced. The changes in the current should vary with varying NO concentrations.

The invention will be further illustrated by the following non-limiting examples.

### EXAMPLES

### Example 1. General Method of Preparation

The electronic properties of semiconductor devices are strongly affected by the properties of the surface, which can be modified by adsorbed molecules. The interaction between the adsorbate and the substrate causes shift of the electron density to or from the surface, depending on the position of the energy state in the adsorbate and the substrate. Thus, the surface charge density and distribution can be changed by the adsorbates, and the effect of the adsorption can be determined.

GaAs is a III-V compound semiconductor with a direct band-gap of 1.42 V. In the experiments herein, GaAs (100) surface was used and the monolayer of the metalloporphyrins was adsorbed on its surface. The adsorption process is monitored using Fourier Transform Infra Red Spectroscopy (FTIR) and X-ray photoelectron Spectroscopy (XPS). As was described above, the metalloporphyrins used have several vibrational bands that are active in the FTIR measurement. The main features are: (1) carbonyl groups, as described above, (2) C═C and C═N bonds from the porphyrin cycle and the exocyclic double bonds, (3) the vibrations arising from the macrocyclic porphyrin system and (4) alkyl substituents.

Organic molecules can be chemically adsorbed on the surface of the GaAs device via several functional groups: phosphates, carboxylic acids, disulfides, thiols, and hydroxamic acids. The best binders are the phosphate and the carboxylic acids, demonstrating irreversible binding under a vast spectrum of conditions. Binding the sensor molecules via a two-site dicarboxylate results in the greater strength of the bonding as compared with sulfides or monocarboxylates. According to the invention we utilized as a non-limiting example naturally occurring porphyrins such as hemin that have two free carboxylic acid groups for illustration of the concept of the invention.

The adsorption of organic compounds having more than one carboxylic acid group proceeds via initial binding of one of the groups and formation of a Ga-carboxylate bond, followed by the adsorption of the second group in the same fashion. At times when the binding domains are in close proximity to each other, the adsorption of the second group may be ineffective because of steric reasons. Differentiation between the two-step adsorption process of dicarboxylic acids and the adsorption process of a single carboxylic acid group was confirmed using both FTIR and electronic measurements.

The IR absorption spectrum of the unbound organic ligand containing a dicarboxylic acid functionality may exhibit peaks corresponding to the symmetric stretching of both carboxylic groups and unsymmetrical stretching that arise from the unequivalent stretching of each group relative to the other. Furthermore, in cases where hydrogen bonding between the carboxylic acid groups is possible, noticeable shifts of the peaks will hint to that. In the IR spectrum of hemin porphyrins the dicarboxylic acid functionality gives rise to a strong and broad band at 1747 cm⁻¹, arising from both the symmetric and unsymmetric vibrations of the two free carboxylic acid groups. The frequency of this band does not attest to any intramolecular hydrogen bonding that may be at play in this molecule.

In the case of a two-step adsorption onto the GaAs surface, two different IR spectra are obtained; one taken 0.5-5 hours after the beginning of the adsorption and the second taken 12 hours thereafter. The differences in the spectra arise from the incomplete adsorption of the dicarboxylic acid functionality to the surface. Four hours after the adsorption begins, only one carboxylic acid (“arm”) is bound to the surface, which is attested to in the IR spectrum by the presence of one carboxylic acid band at around 1740 cm⁻¹ and one Ga-carboxylate band whose frequency is shifted to around 1700 cm⁻¹. The adsorption of the second arm to the GaAs surface requires a longer adsorption time and is observed to end with the nearly complete disappearance of the band at 1740 cm⁻¹ and the strengthening of the 1700 cm⁻¹ band. If steric interactions are not overcome during the longer adsorption times, some bands corresponding to the free carboxylic acid arms may still be present in the IR spectrum.

The MOCSER covered with a monolayer of the metalloporphyrins is introduced into the medium containing nitric oxide molecules. The NO molecules thus bind to the metal centers of the porphyrin monolayer, effecting a change in the electric charge distribution on the surface of the MOCSER. The changes of the current in time are monitored at a constant voltage.

The selectivity of the system towards nitric oxide is evident from the reaction of the metalloporphyrins covered MOCSER with various molecules such as carbon monoxide, carbon dioxide, nitrogen dioxide, oxygen, nitrogen, and water (not shown). The magnitude and the time constant of the change in the current through the MOCSER during exposure to one of the above contaminants is different from the changes in the current during exposure to nitric oxide.

### Example 2. Adsorption of the Metalloporphyrins onto the MOCSER

Prior to each adsorption, the GaAs surface of the device is cleaned by boiling in trichloroethylene, acetone and absolute ethanol for 15 minutes, consecutively, etched for ten seconds in a 1:9 NH₃/H₂O (v/v) solution, washed with de-ionized water and dried under a stream of nitrogen (99.999%). The MOCSERS are then immersed in DMF or CH₃CN solutions containing one of the metalloporphyrins (maximum concentration of 15 mM), for a period allowing maximal adsorption. The devices are next rinsed with 5% chloroform/hexane and blown dry under a stream of nitrogen gas.

In an alternative method, after the etching the MOCSERs are immersed in a 1:1 solution of the metalloporphyrins and benzoic acid. This is done in order to avoid the possible π-π electronic interactions between neighboring porphyrins.

The mixed monolayers are characterized by FTIR using bare, etched, and oxidized GaAs surfaces, as references. The adsorption of the mixed monolayer onto the GaAs results in the appearance of a strong peak at 1710 cm⁻¹ (ν^{as}_{coo}- of porphyrin), while the peaks which are indicative of the free carboxylic acid groups of both the porphyrin and the benzoic acid, at 1747 and 1675 cm⁻¹, respectively, disappear. This indicates that the carboxyl groups bind to the GaAs surface, with a film thickness of about one monolayer (Wu et al., 2000).

AFM images of the mixed monolayer formed indicate that the thickness of the monolayer is about 1.5-1.7 nm, a thickness that is comparable with a monolayer of porphyrins bound through the carboxyl groups and not via stacking. Furthermore, AFM studies indicate that the presence of the benzoic acid molecules assist in forming a more “ventilated” porphyrin monolayer to which the NO approach is facilitated (Wu et al., 2000).

### Example 3. The Measurements

The device response to NO was evaluated at room temperature under anaerobic and aerobic conditions without effecting oxidation of the nitric oxide to the more stable nitrite and nitrate ions.

3.1 Measuring NO Concentrations using NO-Releasing Precursors.

During the experiment, a constant voltage of 100 mV is applied between the ohmic contacts of the MOCSER. The change in the current vs. time, I (t), is monitored in a buffer solution (pH=7.4), while the nitric oxide is released from a precursor such as 1-hydroxy-3-methyl-3-(methylaminopropyl)-2-oxo-1-triazene (t_{½}=10.1 minutes), or other similar triazene compound, at a controllable rate.

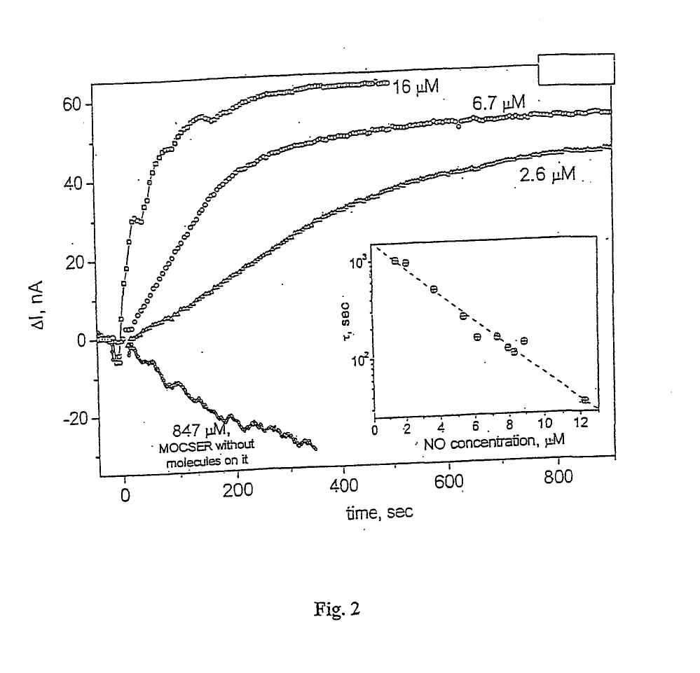

The response of the bare device to high concentrations of nitric oxide is shown in FIG. 2, which represents the response of a typical porphyrin-covered MOCSER to the NO released. The current of the device slightly decreases as compared with the observed increase as a response to the reaction of the nitric oxide with the organic ligand. In addition, unlike the concentration-dependant response observed with the polphyrin-covered device, the response observed with the bare MOCSER is, to a certain extent, concentration independent. From FIG. 2 it is clear that the device's response to the NO produced is rapid, the response is very stable, and current saturation occurs in less than 10 minutes.

Several additional experiments indicate that the response of the MOCSER to NO results solely from the interaction of the organic monolayer with varying concentrations of NO, and that there was no measurable response to the following: 1) solutions of the NO-releasing precursors prepared under conditions such that the NO molecules are not produced; 2) buffer solutions (pH=7.4) containing none of the NO precursor; 3) solutions at pH=10-11; 4) solutions of the metabolites produced from the NO-producing precursors (diamines); and 5) porphyrin systems containing no metal center.

3.2 Measuring NO in Hippocampal Slices in Artificial Cerebral Spinal Fluid (ACSF).

Brain slices of rat or guinea pig release NO after depolarization induced by high potassium or after electrical stimulation of the slice, but the production of H₂O₂ is unavoidable. The response of the bare MOCSER to hydrogen peroxide arises from the oxidation of the device's surface. However, when the surface of the MOCSER is covered with a monolayer of organic compounds, the reactivity of the GaAs surface reduces dramatically. A differentiation between the response towards the hydrogen peroxide and the nitric oxide both evolved in the process of brain cell stimulation is possible due to the successful protection of the GaAs surface by the porphyrin monolayer.

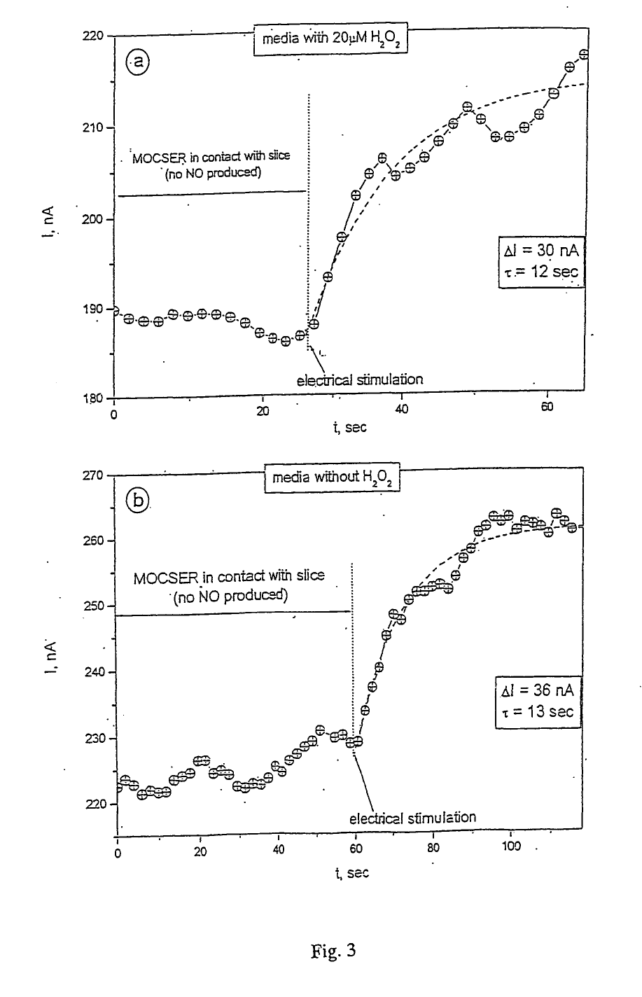

The measurements were performed in the presence and absence of 20 μM of hydrogen peroxide. Electrical stimulation of the brain slices (one-second train of pulses at a rate of 100 Hz) was started after the MOCSER was in prolonged and continuous contact with the slice and the media, and after signal stabilization (base line). FIGS. 3a-3b show measurements of NO produced by brain tissues as measured by a MOCSER immersed in the artificial cerebrospinal fluid (ACSF) at a distance of less than 1 mm from the brain slice, in the presence (FIG. 3a) and absence (FIG. 3b) of H₂O₂.

As FIGS. 3a and 3b show, the MOCSER immersed in the ACSF, at a distance of less than 1 mm from the brain slice, showed no detectable response towards hydrogen peroxide prior to or after electrical stimulation. The response observed arises solely from the evolution of NO. There is an increase of the current as a result of the slice stimulation.

Two parameters were extracted from each of the measurements: the amplitude, ΔI, of the current change (difference between the current saturation and the initial current prior to stimulation) and the time constant, τ, characterizing the rate of the NO binding to the MOCSER. The observed values of ΔI are 30-80 nA which correspond to a concentration of several μM of NO.

In these measurements, the release of NO from the brain slices depends on the response to the electrical stimulation. This allows a supply of NO to the media in one batch and without further replenishment. With measurements utilizing the NO-releasing precursors (see above), the time constant is controlled by the rate at which the NO is released from the organic precursors. In the brain slices measurements τ is dependent on the NO decomposition process, meaning on its half-life. Therefore, the two time constants namely, of NO released from brain slices and of NO released from the NO-releasing precursors, are not comparable and do not define an identical process. The processes that bring about the NO-porphyrin binding are fast relative to the other processes and can thus be neglected. In fact, the observed τ values are 12-13 seconds, which correspond nicely with the reported nitric oxide half-life of about 5-15 seconds.

3.3 Measuring Gaseous NO Concentrations.

Gas mixtures of NO in nitrogen gas or, alternatively, in dry air (containing 79% nitrogen, 21% oxygen, 530 ppm CO₂, 5 ppm CO and <6 ppm H₂O) were prepared in various concentrations, varying from 5 ppb to 10 ppm NO in N₂ or air, using a Multi-Gas Calibrator. Each gas mixture was brought in contact with the MOCSER at a constant flow, temperature and under controlled consistent conditions. A constant voltage of 100 mV was applied to the MOCSER and a current flowing through the MOCSER was monitored using a Source-Measuring Unit.

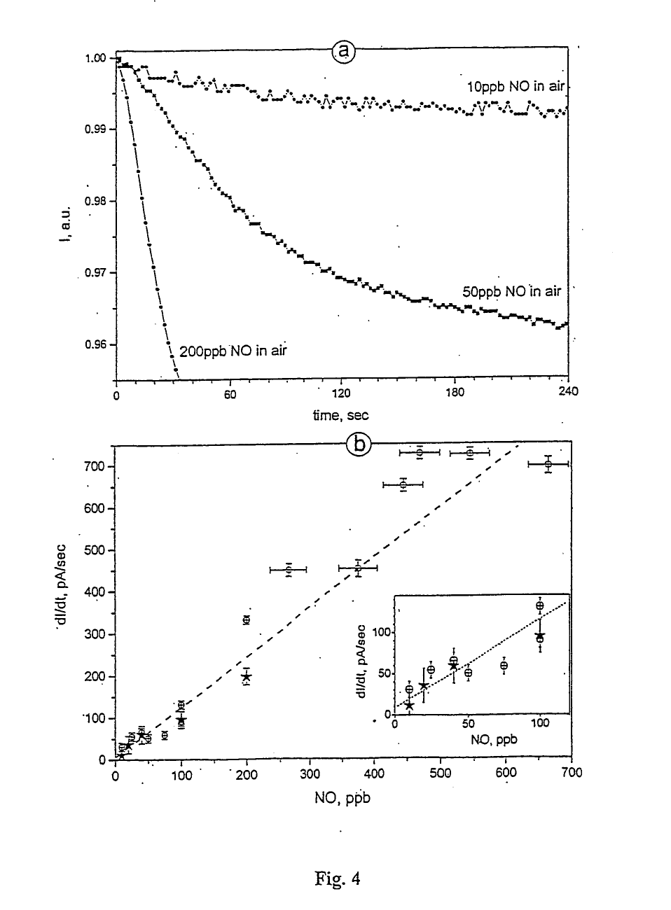

The sensitivity of the sensors, covered by a monolayer of Cobaltic Protoporphyrin IX, to the NO is shown in FIG. 4a. The varying concentrations produced consistent and reliable responses that allowed facile differentiation of NO concentrations. As can be seen from FIG. 4a, the electrical current decreased significantly when the sensor was exposed to NO. The response of the device depended on the concentration of NO and its reproducibility in a constant concentration of NO was excellent as tested on a single device or on different ones. Both the saturation value of the current change ΔI=(Iₛₐₜᵤᵣₐₜᵢₒₙ-Iₒ) and the rate of the current change dI/dt correlate with the NO concentration (FIG. 4a), therefore, both parameters can be used for the sensor calibration. The calibration curve shown in FIG. 4b presents the dependence of dI/dt on the NO concentration in the range 0-700 ppb both in nitrogen (shown by open circles) and in dry air (shown by stars) for MOCSERs, covered by a monolayer of Cobaltic Protoporphyrin IX. There is no significant difference between the calibration curves obtained in nitrogen and in dry air that demonstrates that the sensitivity of the sensor to NO is not influenced by the presence of oxygen, CO₂ and CO.

Only a weak, almost concentration-independent, response of the MOCSER to NO was observed in the absence of the organic porphyrin or the organic porphyrin-benzoic acid mixed monolayer that confirmed that NO interactions were with the organic monolayer that, in turn, influenced the GaAs surface.

### Example 4. Reversibility of the NO Sensor

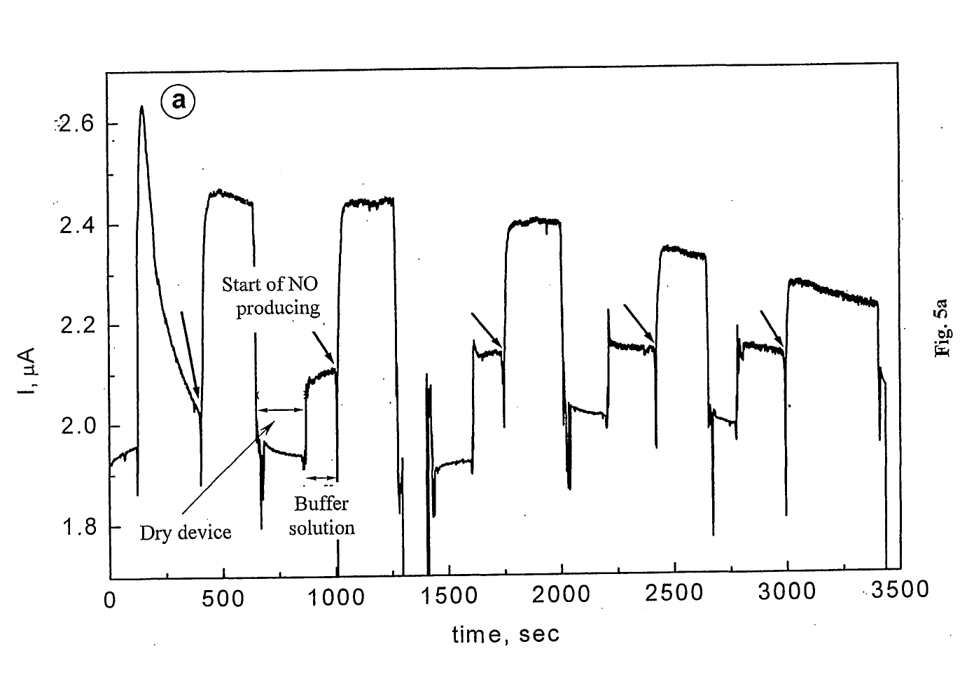

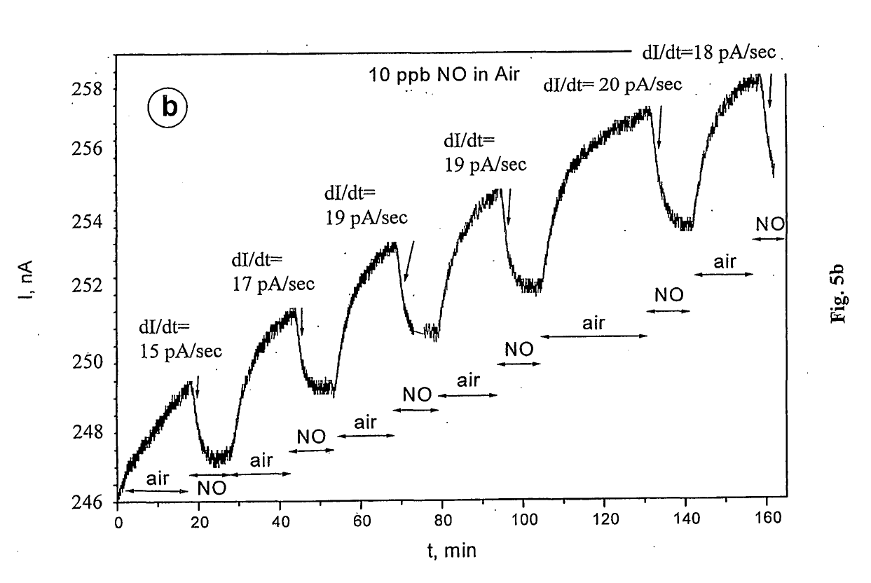

The reversibility and usability of the MOCSER as a sensor for nitric oxide was demonstrated in both the aqueous and gas phases. Over a cycle of several measurements the sensor was exposed to the NO-containing medium (gas or solution), taken out and purged with nitrogen gas or dry air and measured again. In aqueous solution, the saturated current relative to the original change are 1:0.74:0.57:0.44:0.31 (FIG. 5a), demonstrating a reasonable reversibility of the system. The decrease of the saturated current upon repeated cycling indicates that the porphyrin layer is either slowly oxidized or damaged. In gas mixtures (FIG. 5b), the effect of the deterioration of the sensor sensitivity was much weaker, demonstrating the same rates (18±2 pA/sec) of the change in the current over a cycle of several measurements. From FIG. 5b it is clear that the device can be regenerated for further and continuous use by nitrogen gas or dry air purge profile. The purging period results in a complete regeneration of the response once the same device was re-exposed to the same NO concentration. In addition, exposing the NO-bound layer to a short laser pulse (50 ns, 532 nm) regenerates the NO-free monolayer.

### Example 5. Sensitivity of the Device to Nitric Oxide

5.1 Liquid Phase

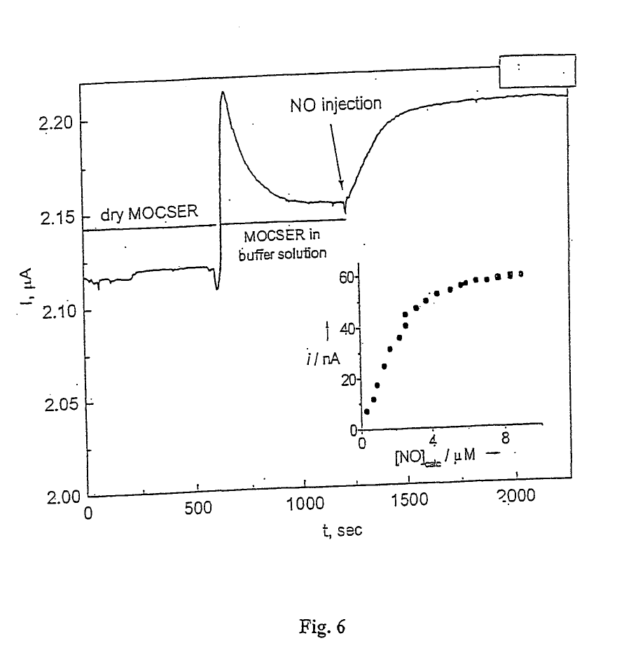

From FIG. 6 it is apparent that the device is highly sensitive to NO produced in vitro and that response is quite rapid. From direct measurements it was found that the device is sensitive to concentrations as low as 1.3 μM (39 ppb; 1 μM=30 ppb NO) in solution. It is also worth of noting that the response time of the current is different at different NO concentrations. This is shown in FIG. 2: when the current reaches “steady-state”the response time is about 5, 10, and 20 minutes for concentrations of 16, 6.7, and 2.6 μM (480, 210 and 78 ppb) of NO-releasing solution, respectively.

In order to understand the sensitivity of the device, the concentration of NO was measured at different times (from FIG. 6) using the equation:

[NO]=C₀(1−e^{-1.16×10-3t})

where [NO] is the concentration of NO at time, t(sec), and C₀ is the total concentration of the NO adduct in the buffer solution. The relationship between the current and the concentration that is obtained is shown in FIG. 6. From this, it can be concluded that the device can respond to NO concentrations of as low as 0.7 μM.

Experimentally, we find a correlation for the change in the current upon introduction of the NO medium over a certain time range. From the known variables t and C₀, we can calculate the exact concentration of NO at any time for which the linear correlation holds (Wu et al., 2000).

5.2 Gaseous Phase

FIG. 4 demonstrates the directly measured responses of the sensor to the NO concentrations down to 10 ppb. The response is quite rapid: the period of 10-20 sec is enough in order to distinguish between the responses to different NO concentrations and to calculate the rate of the current change,

$$\frac{ⅆ I}{ⅆ t} ,$$

accurately. It is clear from the response of the sensor to the concentration of 10 ppb that the signal-to-noise ratio is rather good and allows determination of even lower concentrations of NO.

### Example 6. Calibration of the Device to NO

The device is calibrated to report accurate concentrations of NO in the examined media. The calibration curves utilized are based on series of measurements of varying concentrations of NO. Each media produces a different calibration curve, as can be seen in the inserts of FIGS. 2 and 4.

### Example 7. Sensitivity and Selectivity of the NO Sensor for other Substances

As was shown earlier, the response of the device in a medium containing NO stems solely from interactions between the porphyrin monolayer and the NO present. Experiments with each component of the various media or various mixtures thereof resulted in no response from the device. In this aspect, the bare MOCSER or the MOCSER covered with a monolayer of porphyrin molecules exhibited no detectable response to water, buffer solutions over a range of pH values, to solutions of free amines and ammonium salts, or to NO-releasing compounds or their metabolites (not shown).

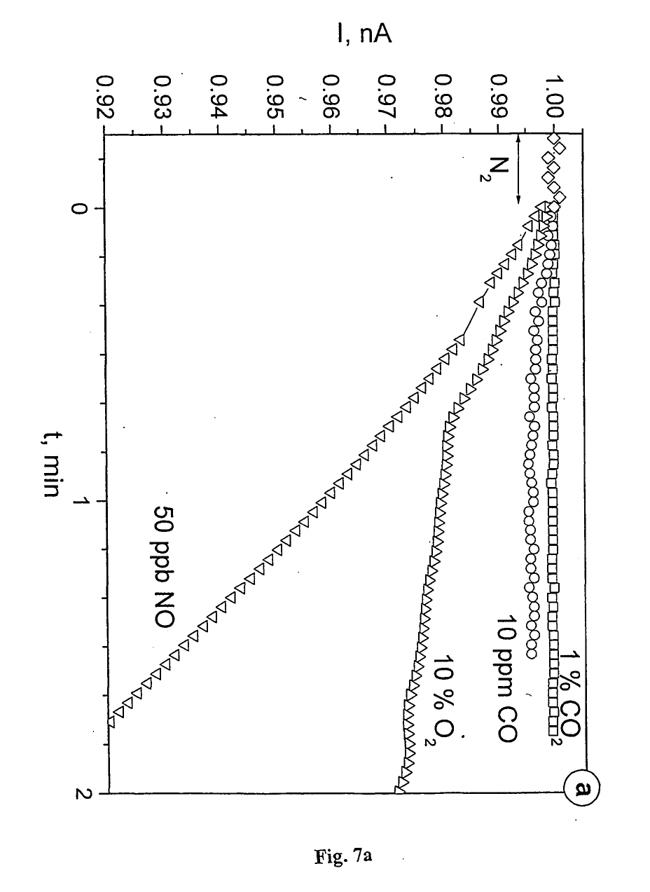

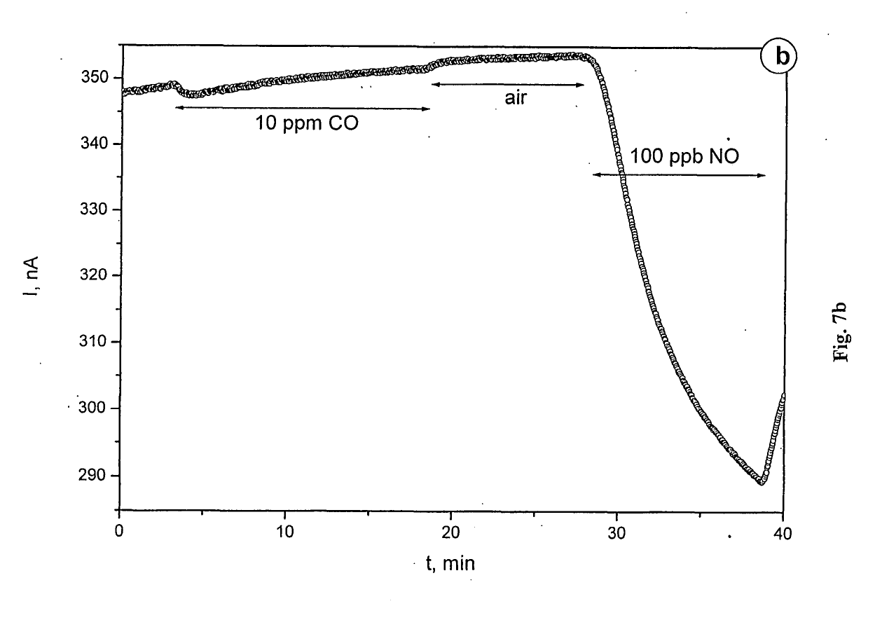

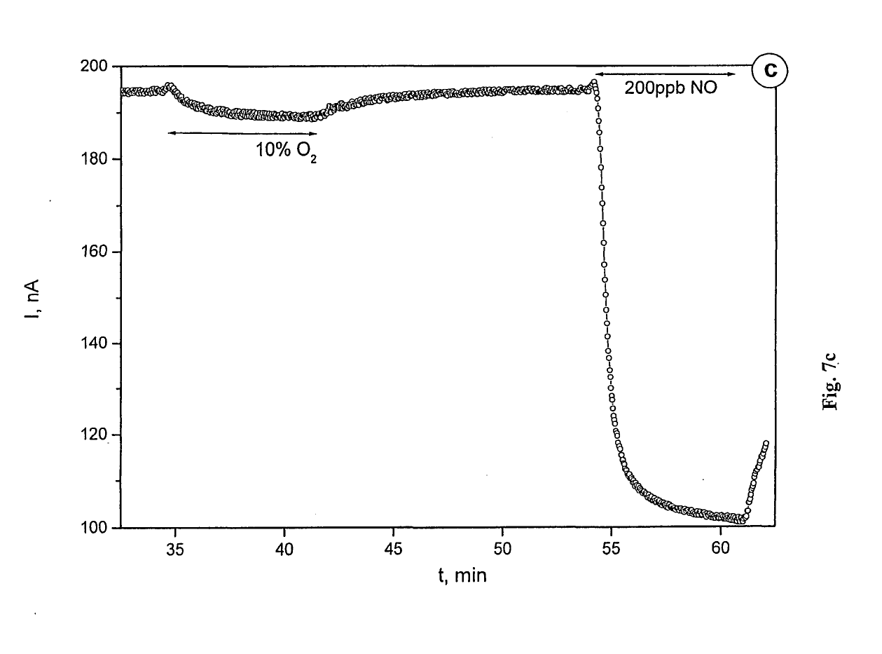

FIG. 7 demonstrates the selectivity of the NO sensor towards gaseous substances. As can be seen from the FIG. 7a the response of the device towards 10 ppm carbon monoxide in nitrogen and 1% carbon dioxide in nitrogen is minor. Although the response of the sensor towards 10% oxygen in nitrogen is significant, the comparability of the NO calibration curves, obtained with nitrogen or dry air (containing 21% oxygen) as a diluting gas (see FIG. 4b), proves that the sensitivity of the sensor to NO is not affected by the presence of oxygen. There is no detectable response of the sensor to other inert gases.

Pre-exposition of the sensor to different gases before the exposure to NO does not affect the sensitivity of the sensor towards NO. As can be seen from the FIG. 7b and FIG. 7c, the response of the sensor, exposed to 10 ppm carbon monoxide or 10% oxygen and purged with nitrogen or dry air afterwards, is similar to that of a non-used device. That proves the high selectivity of the organic monolayer towards NO in presence of much higher concentrations of different gases.

### Example 8. Contact Potential Difference (CPD) Measurements

Kelvin probe measurements were performed in order to study the effects of the adsorbed porphyrin molecules on the device's electronic properties. The 1:1 mixture of porphyrin and benzoic acid was adsorbed on the GaAs surface of the MOCSER, as was described earlier, and the contact potential difference (CPD) between the n-GaAs surface and the Au grid was measured by a Kelvin probe in ambient.

The effective electron affinity (χ) was found to increase as a result of the porphyrin adsorption onto the GaAs surface, which also caused a decrease in the band bending (Vₛ) of the sample studied (not shown). For example, for bare n-GaAs χ=4.4±0.2 V and Vₛ=350±40 mV, while after adsorption of the porphyrin χ=4.6±0.2 V and Vₛ=320±80 mV. This change indicates that the dipole of the adsorbed molecules is oriented with the negative pole pointing away from the surface with a minor decrease of the net surface charge.

Discussion

Nitric Oxide (NO) is recognized as playing a crucial role in a vast number of functions in mammalian life. The basic requirement for the development of a diagnostic tool for measuring NO is the development of a cheap and reliable sensor.

According to the invention we showed that the MOCSER in its current embodiment could be successfully used as a sensor for the detection of nitric oxide in biological media, in gas mixtures and in aqueous media. The sensitivity of the device described here towards NO is independent of other species present in the tested medium. Furthermore, unlike other NO sensors described in the literature, the device based on MOCSER is easy and cheap to manufacture, manipulate, and operate.

On the basis of the IR spectra it is clear that a sufficient monolayer of porphyrin molecules is formed on the surface of the GaAs based device. The binding that occurs via a set of two carboxylic acid groups is achieved in a homogeneous solution of the porphyrin in DMF. The binding and stability were monitored and studied by FTIR, XPS and CPD measurements.

With the presented device, three different media containing varying NO concentrations were examined. The different threshold sensitivity to low concentrations of NO observed for the three media arises from the dynamics of the NO approach to the sensor molecules.

In solution media concentrations of as low as 30 ppb NO are detected in the presence of other dissolved organic and inorganic compounds, such as hydrogen peroxide, free amines, ammonium salts, hydrocarbons, and dissolved gases. In gaseous media, concentrations of as low as 10 ppb were detected with selectivity of several orders of magnitude towards other gases. This unique selectivity of the porphyrin layer and even more importantly the ability of the device to electrically distinguish between various species give the sensor of the invention its powerful characteristics.

Reusability of the sensor is another aspect that is of importance. The MOCSER device may be reused over time by simply purging the surface of the device with nitrogen gas or dry air. The devices are stable in inert atmosphere and at room temperature for long periods of time (several months). This is important for the construction of sensors that can be stored for long periods of time.

All of the above mentioned characteristics of the sensor device afford a system with manifold potential applications.

References Becker Th., Muhlberger St., Braunmuhl Chr. Bosch-v., Muller G., Ziemann Th., Hechtenberg K. V., “Air pollution Monitoring Using Tin-Oxide Based Microreactor Systems”, Sensors and Actuators B, 69, 108-119 (2000).

Casey T. E., Hildelman R. H., “Modification of the Cadmium Reduction Assay for Detection of Nitrite Production Using Fluorescence Indicator 2,3-Diaminonaphthalene”, Nitric Oxide: Biology and Chemistry, 4, 67-74 (2000).

Gartsman K., Cahen D., Kadyshevitch A., Libman J., Moav T., Naaman R., Shanzer A., Umansky V., Vilan A., “Molecular Control of a GaAs Transistor”, Chem. Phys. Lett., 283, 301 (1998).

Hunt J., Gaston B., “Airway Nitrogen Oxide Measurements in Asthma and Other Pediatric Respiratory Diseases”, J. Pediatr., 137, 14-20 (2000).

Kissoon N., Duckworth L., Blake K., Murphy S., Silkoff P. E., “Exhaled Nitric Oxide Measurements in Childhood Asthma: Techniques and Interpretation”, Ped. Pulm., 28, 282-296 (1999).

Kojima H, Nagano T., “Fluorescent Indicators for Nitric Oxide”, Adv. Mater., 12, 763-765 (2000).

Kotake Y., Moore D R., Sang H., Reinke L. A., “Continuous Monitoring of in Vivo Nitric Oxide Formation Using EPR Analysis in Biliary Flow”, Nitric Oxide: Biology and Chemistry, 3, 114-122 (1999).

Kudo M., Kosaka T., Takahashi Y., Kokusen H., Sotani N., Hasegawa S., “Sensing Functions to NO and O₂ of Nb₂O₅— or Ta₂O₅-Loaded TiO₂ and ZnO”, Sensors and Actuators B, 69, 10-15 (2000).

Marzinzig M, Nussler A. K, Stadler J, Marzinzig E, Barthlen W, Nussler N. C, Beger H. G, Moris S. M, Bruckner U. B., “Improved Methods to Measure End Products of Nitric Oxide in Biological Fluids: Nitrite, Nitrate, and S-Nitrosothiols”, Nitric Oxide: Biology and Chemistry, 1, 177-189 (1997).

Menil F., Coillard V., Lucat C., “Critical Review of nitrogen Monoxide Sensors for exhaust Gases of Lean Burn Engines”, Sensors and Actuators B, 67, 1-23, (2000).

Schulz K., Kerber S., Kelm M., “Reevaluation of the Griess Method for determining NO/NO₂⁻ in Aqueous and Protein-Containing Samples”, Nitric Oxide: Biology and Chemistry, 3, 225-234 (1999).

Vilan A., Ussyshkin V. R., Gartsman K., Cahen D., Naaman R., Shanzer A., “Real Time Monitoring of Adsorption Kinetics: Evidence for 2-site Adsorption Mechanism of Dicarboxylic Acids on GaAs (100)”, J. Phys. Chem. (B), 102, 3307-3309 (1998).

Wang X., Miura N., Yamazoe N., “Study of WO₃-Based Sensing Materials for NH₃ and NO Detection”, Sensors and Actuators B, 66, 74-76 (2000).

Weitzbarg E. Lundberg J. O. N., “Nonenzymatic Nitric Oxide Production in Humans”, Nitric Oxide: Biology and Chemistry, 2, 1-7 (1998).

Wu D. G., Ashkenasy G., Shvarts D., Ussyshkin V. R., Naaman R., Shanzer A., Cahen D., “Novel NO Biosensor, Based on Surface Derivatization of GaAs by ‘Hinged’Iron-Porphyrins”, Angew. Chem., Int. Ed. Engl., 39 (24), 4496-4500 (2000).

Wu D. G., Cahen D., Graf P., Naaman R., Shanzer A., Shvarts D., “Direct Detection of Low Concentration NO in a Physiological Solutions by a New GaAs Based Sensor”, Chem. Eur. J., 7(8), 1743-1749 (2001).

[image]

[image]

1. A semiconductor device for the detection of Nitric Oxide (NO), said device being composed of:
(i) at least one layer of a conducting semiconductor;
(ii) at least one insulating or semi-insulating layer;
(iii) a layer of multifunctional organic molecules capable of binding nitric oxide (NO), said molecules being directly bound to the surface of an upper layer which is either a conducting semiconductor layer (i) or an insulating or semi-insulating layer (ii); and
(iv) two conducting pads on the top layer making electrical contact with the electrically conducting layer (i), such that electrical current can flow between them at a finite distance from the surface of the device.

2. A semiconductor device according to claim 1 for the detection of nitric oxide (NO), said device being composed of one or more insulating or semi-insulating layers (1), one conducting semiconductor layer (2), two conducting pads (3), and a monolayer of multifunctional organic molecules capable of binding NO (4), characterized in that:
said conducting semiconductor layer (2) is on top of one of said insulating or semi-insulating layers (1),
said two conducting pads (3) are on both sides on top of an upper layer which is either said conducting semiconductor (2) or another of said insulating or semi-insulating layers (1), making electrical contact with said conducting semiconductor (2),
and said monolayer of multifunctional organic molecules capable of binding NO (4) is adsorbed on the surface of said upper layer, between the two conducting pads (3).

3. A semiconductor device according to claims 1 or 2, wherein said multifunctional organic molecules of layer (iii) that can bind NO are selected from vicinal diamines, metalloporphyrins, metallophthalocyanines, and Iron-dithiocarbamate complexes that contain at least one functional group selected from carboxyl, thiol, acyclic sulfide, cyclic disulfide, hydroxamic acid, trichlorosilane or phosphate.

4. A semiconductor device according to claim 3, wherein said vicinal diamine that binds NO is 2,3-diaminonaphthalene, 1,2-diaminobenzene, 1,2-diaminoanthraquinone or aminotroponiminate that are substituted at at least one of the amino groups with one suitable functional group selected from carboxyl, thiol, acyclic sulfide, cyclic disulfide, hydroxamic acid, trichlorosilane or phosphate, linked to the amino group through an aliphatic, aromatic or araliphatic spacer.

5. A semiconductor device according to claim 4, wherein said vicinal diamine that binds NO is 2,3-diaminonaphthalene.

6. A semiconductor device according to claim 4, wherein said vicinal diamine that binds NO is 1,2-diaminobenzene.

7. A semiconductor device according to claim 3, wherein said metalloporphyrin or metallophthalocyanine that binds NO contains as central atoms a metal atom selected from Fe, Co, Ni, Zn, Mn, Cu, Ru, V, Pb, or Cr.

8. A semiconductor device according to claim 7, wherein said metalloporphyrin is derived from hematoporphyrin or protoporphyrin IX.

9. A semiconductor device according to claim 8, wherein said metalloprotoporphyrin IX is hematin (ferriprotoporphyrin basic), heme (ferroprotoporphyrin), hemin (ferriprotoporphyrin chloride) or cobaltic protoporphyrin IX chloride.

10. A semiconductor device according to claim 3, wherein said NO-binding compound is an iron-dithiocarbamate complex.

11. A semiconductor device according to any one of claims 1-10, wherein said conducting semiconductor layer (2) is a semiconductor selected from a III-V and a II-VI material, or mixtures thereof, wherein III, V, II and VI denote the Periodic Table elements III=Ga, In; V=As, P; II=Cd, Zn; VI=S, Se, Te.

12. A semiconductor device according to any of claims 1-11, wherein said conducting semiconductor layer (2) is doped n-GaAs or doped n-(Al,Ga)As.

13. A semiconductor device according to any one of claims 1-12, wherein the one or more insulating or semi-insulating layers (1), that may serve as the base for the device, is a dielectric material selected from the group consisting of silicon oxide, silicon nitride and an undoped semiconductor selected from a III-V and a II-VI material, or mixtures thereof, wherein III, V, II and VI denote the Periodic Table elements III=Ga, In; V=As, P; II=Cd, Zn; VI=S, Se, Te.

14. A semiconductor device according to claim 13, wherein said undoped semiconductor is undoped GaAs or undoped (Al,Ga)As.

15. A semiconductor device according to any of claims 1-14, wherein said conducting semiconductor layer (2) of doped n-GaAs is on top of a semi-insulating layer (1) of (Al,Ga)As which is on top of another semi-insulating layer (1) of GaAs, and on top of said conducting semiconductor doped n-GaAs layer (2) there is a semi-insulating undoped GaAs layer (1) to which is attached a monolayer of said NO-binding molecules (4).

16. A semiconductor device according to any one of claims 1-14, wherein said conducting semiconductor layer (2) of doped n-(Al,Ga)As is on top of an insulating layer (1) of undoped GaAs which is on top of a semi-insulating layer (1) of GaAs, on top of said conducting doped n-(Al,Ga)As layer (2) there is a semi-insulating undoped (Al,Ga)As layer (1) on top of which there is an upper undoped GaAs semi-insulating layer (1), and said monolayer of NO-binding molecules (4) is attached to the upper undoped GaAs semi-insulating layer (1).

17. A semiconductor device according to any of claims 1-16, wherein said monolayer of NO-binding molecules further comprises benzoic acid molecules.

18. An array of semiconductor devices according to any one of claims 1-17, wherein each device in the array is covered with a monolayer consisting of a different NO-binding molecule.

19. An array of semiconductor devices according to any one of claim 1-18, wherein at least one of the said devices carries a monolayer of a NO-sensitive molecule and other devices in the array carry monolayers comprised of compounds capable to bind to contaminants of NO mixtures.

20. A method for the detection and measurement of nitric oxide, which comprises:
(i) exposing the semiconductor device according to any one of claims 1-17 or an array of devices according to claim 18 or 19, to a sample containing NO; and
(ii) monitoring the presence of NO in the sample and determining its concentration according to the change in the current measured at a constant electric potential applied between the two conducting pads.

21. A method according to claim 20 wherein said sample is gaseous, aqueous or mixtures thereof.

22. A method according to claim 20 or 21 wherein said sample is a biological fluid.

23. A method according to claim 22 wherein said biological fluid is exhaled air.

24. A method according to claim 22 wherein said biological fluid is endogenous gaseous NO of the urogenital tract.

25. A method according to claim 22 wherein said biological fluid is endogenous gaseous NO from the lumen of the intestines.
